(19) United States
(12) Patent Application Publication
(10) Pub. No.: US 2008/0058954 A1
(43) Pub. Date: Mar. 6, 2008

(54) Methods of treating spinal injuries using injectable flowable compositions comprising organic materials

(76) Inventor: Hai Trieu, Cordova, TN (US)

Correspondence Address: FOX ROTHSCHILD, LLP, 997 LENOX DRIVE, LAWRENCEVILLE, NJ, 08648, US

(21) Appl. No.: 11/507,682

(22) Filed: Aug. 22, 2006

(51) Int. Cl. A61F 2/28

(52) U.S. Cl. 623/23.62

(57) ABSTRACT

The instant invention discloses novel methods of treating vertebrae, intervertebral discs, and facet joints by administering to these areas or to the implants in these areas a flowable composition comprising an organic material capable of being cured or polymerized in vivo.

### FIELD OF THE INVENTION

The present invention provides a method of using flowable and injectable organic materials for therapeutic in vivo uses for spinal applications concerning diseased or collapsed vertebra bodies, degenerative intervertebral discs, degenerative facet joints, post surgical adhesion prevention in spinal areas having hard or soft tissues, and various spinal devices.

### BACKGROUND

The spine is formed by a plurality of stacked irregular bones called vertebrae. Each vertebra is individually separated by a intervertebral disc and connected to other vertebrae by facet joints. The vertebrae of the spine, in concert with the intervertebral discs and the facet joints, provide a remarkably strong and flexible structure capable of withstanding substantial forces.

However, vertebral fractures can occur when diseases weaken vertebral bone tissue or are collapsed by traumatic forces. Vertebral fractures are commonly caused by osteoporosis. This disease is characterized by low bone mass and structural deterioration of bone tissue, leading to bone fragility and an increased susceptibility to vertebral fractures.

Vertebra compression fractures from trauma often require rigid bracing to protect the bone as it heals. If bone tissue migrates into the spinal canal, spinal surgery may be required to remove the bone tissue in addition to further surgery to stabilize the collapsed vertebra by spinal fusion procedures. Morbidity associated with vertebrae compression fractures are continued pain, limited flexibility, and spinal deformity.

In lieu of surgery, minimally invasive techniques can help alleviate the pain of compression fractures. Vertebroplasty is one such process and is generally accomplished by injecting polymethylmethacrylate (PMMA) cement into the vertebral body through a needle into the fractured bone. While PMMA has high mechanical strength, it cures fast and thus allows only a short handling time. Other potential problems of using PMMA injection include damage to surrounding tissues by a high polymerization temperature or by the unreacted toxic monomer, and the lack of long-term biocompatibility.

The vertebrae are separated by intervertebral discs which are anterior to the spinal cord. Each intervertebral disc has a relatively tough outer layer called the annulus fibrosus that surrounds a gel-like inner layer called the nucleus pulposus. The discs have several functions, one of which includes serving as shock absorbers for the vertebrae.

Intervertebral discs may be displaced or damaged due to trauma or disease. Disruption of the annulus fibrosis allows the nucleus pulposus to protrude into the spinal canal, a condition commonly referred to as a herniated or ruptured disc. The extruded nucleus pulposus may press on the spinal nerve, which may result in nerve damage, pain, numbness, muscle weakness and paralysis. Intervertebral discs may also deteriorate due to the normal aging process. As a disc dehydrates and hardens, the disc space height will be reduced, leading to instability of the spine, decreased mobility and pain.

One way to relieve the symptoms of these conditions is by surgical removal of a portion or all of the intervertebral disc. The removal of the damaged or unhealthy disc may allow the disc space to collapse, which could lead to instability of the spine, abnormal joint mechanics, nerve damage, as well as severe pain.

Several devices exist to fill an intervertebral space following removal of all or part of the intervertebral disc in order to prevent disc space collapse or to promote fusion of adjacent vertebrae surrounding the disc space. Even though a certain degree of success with these devices has been achieved, full motion is typically never regained after such intervertebral fusions.

Facet joints link vertebrae together and provide mobility and support for the spine. There are two facet joints between each pair of vertebrae. The surfaces of the facet joints are lubricated with an inner fluid that allows vertebral bones to glide against each other with little resistance. A watertight sac of soft tissue and ligaments contains the fluid and encloses the facet joint.

Degradation of facet joints occurs when cartilage in the joints are worn down as a result of wear and tear, aging, injury or misuse. Laser Facet Thermal Ablation is a method of treating the pain associated with worn facet joints. This procedure is performed via the insertion of a small tube into the damaged facet joint. The laser is collimated and optically focused onto the damaged facet joint. The laser is used to clean the joint and deaden the nerve that innervates the joint.

The monetary cost of treating vertebral fractures and defects of facet joints and intervertebral discs are enormous is likely to increase with increased longevity of the population. For example, 700,000 vertebral fractures occur each year in the United States. The number of people in the United States aged 65 or more is expected to more than double from 32 million in 1990 to 69 million in 2050. Those aged 85 years or more are expected to increase 5-fold from 3 million to 15 million. Direct medical expenses for osteoporotic fractures alone were estimated at $13.8 billion in 1995.

Accordingly, there is a need for minimally invasive treatments for spinal bone and joint repair.

### SUMMARY OF INVENTION

The instant invention addresses these and other needs by providing novel methods of bone and joint repair using injectable materials.

In one aspect, the invention provides a method of treating a patient having a fractured vertebra comprising administering to said fractured vertebra a flowable composition comprising an organic material, capable of being cured or polymerized into a solid substance in vivo, said solid substance having an elastic modulus of at least about 100 MPa, wherein at least about 50% of said solid substance is bioresorbed or biodegraded within 10 years from the curing or polymerization.

In another aspect, the invention provides a method of treating a patient having a back pain caused by a degenerative facet joint or a degenerative disc disease comprising administering into the degenerative facet joint or the degenerating disc a flowable composition comprising an organic material, capable of being cured or polymerized into a solid substance in vivo, said solid substance having an elastic modulus of at least about 1 MPa, wherein at least about 50% of said solid substance is bioresorbed or biodegraded within 10 years from the curing or polymerization. In one embodiment, the method does not require any removal of tissue (e.g., a disc tissue or a facet joint tissue) prior to the administration of the composition.

In another aspect, the invention provides a method of preventing an adhesion of a first tissue to at least a second tissue after a surgery comprising administering to the first tissue a flowable composition comprising an organic material, capable of being cured or polymerized into a film in vivo, said film having an elastic modulus of at least about 50 MPa, wherein at least about 50% of said film is bioresorbed or biodegraded within 10 years from the curing or polymerization.

In yet another aspect, the invention provides a method of tissue augmentation comprising administering to a tissue in need thereof a flowable composition comprising an organic material, capable of being cured or polymerized into a solid substance in vivo, said solid substance having an elastic modulus of at least about 1 MPa, wherein at least about 50% of said solid substance is bioresorbed or biodegraded within 10 years from the curing or polymerization.

In yet another aspect, the invention provides a method of treating a tissue defect comprising inserting an inflatable device into the tissue defect; and inflating the inflatable device with a flowable composition comprising an organic material, capable of being cured or polymerized into a solid substance in vivo, said solid substance having an elastic modulus of at least about 5 MPa, wherein at least about 50% of said solid substance is bioresorbed or biodegraded within 10 years from the curing or polymerization.

In different embodiments of the invention, the composition may further comprise at least one additive. In different embodiments of the invention, the at least one additive is selected from the group consisting of growth factors, analgetics, anesthetics, antibiotics, anti-inflammatory agents, radiocontrast agents, cells, compounds inducing cell attachment, compounds suppressing cell attachment, and any combinations thereof.

### DESCRIPTION OF PREFERRED EMBODIMENTS

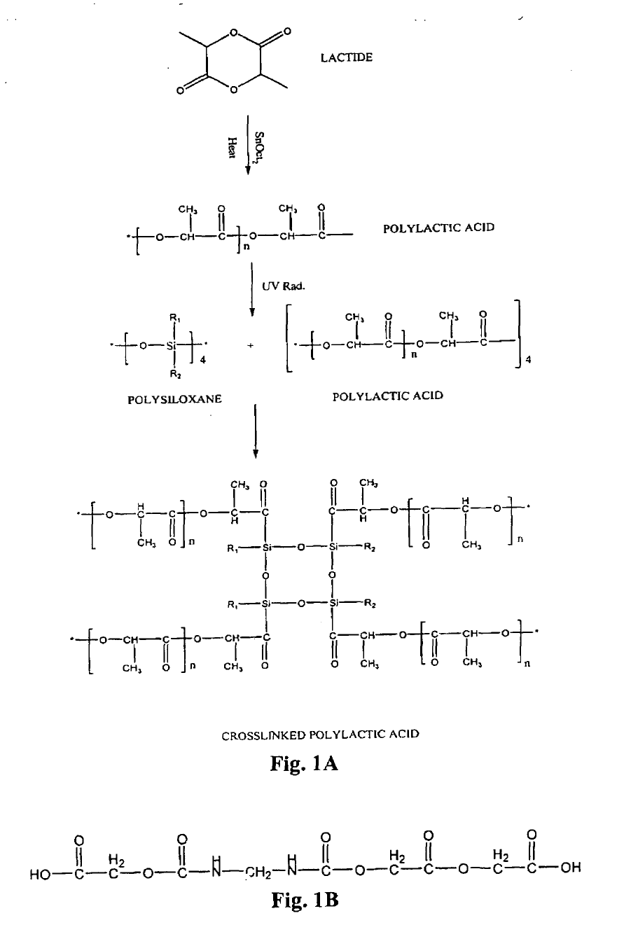

FIGS. 1A and 1B illustrate different embodiments of the flowable composition and the resulting solid body.

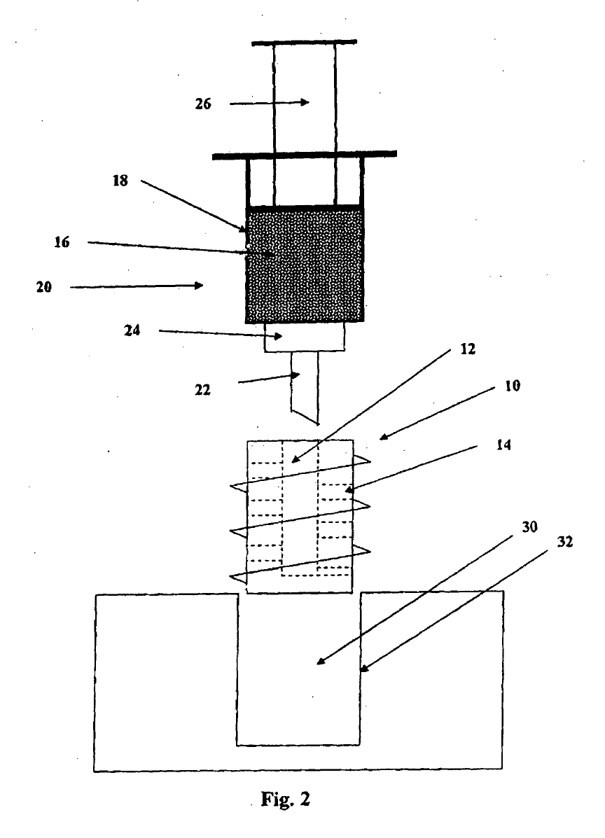

FIG. 2 illustrates the use of the flowable composition inside and outside of an implant.

### DESCRIPTION OF PREFERRED EMBODIMENTS

For the purposes of promoting an understanding of the principles of the invention, reference will now be made to preferred embodiments and specific language will be used to describe the same. It will nevertheless be understood that no limitation of the scope of the invention is thereby intended, such alterations and further modifications of the invention, and such further applications of the principles of the invention as illustrated herein, being contemplated as would normally occur to one skilled in the art to which the invention relates.

Definitions

To aid in the understanding of the invention, the following non-limiting definitions are provided:

“Essentially non-porous” substance refers to a substance having pores which do not have a diameter sufficient to support tissue ingrowth but allow fluid interchange across that substance. The term also refers to the substance having voids or pores comprising less than about 5% of the volume of the substance.

The term “bioresorbable” refers to an ability of being metabolized by the body. Thus, a bioresorbable material loses its mass due to body metabolism.

The term “biodegradable” refers to loss of mechanical properties of a material. Thus, for example “50% biodegradable” material refers to a loss of 50% of the material's mechanical properties (e.g., mechanical strength).

The term “additive” refers to any molecule, cell, intracellular structure, or any combination thereof. As a way of a non-limiting example, both a molecule, such as, for example, rhBMP-2, and a cell, such as, for example, a stem cell, are included within the meaning of the term “additive.”

The term “microspheres” refers to generally spherical particles 10 μm-100 μm in size. Microspheres may comprise, for example, a hollow space encapsulated by lipids, polymers, at least one surfactant, or any combination thereof, wherein the hollow space comprises therapeutic agent, such as at least one additive. In different embodiments, microspheres may include microbubbles and liposomes.

The term “subject” shall mean any animal belonging to phylum Chordata, including, without limitation, humans.

The term “treating” or “treatment” of a disease refers to executing a protocol, which may include administering one or more drugs to a subject (human or otherwise), in an effort to alleviate signs or symptoms of the disease. Alleviation can occur prior to signs or symptoms of the disease appearing, as well as after their appearance. Thus, “treating” or “treatment” includes “preventing” or “prevention” of disease. In addition, “treating” or “treatment” does not require complete alleviation of signs or symptoms, does not require a cure, and specifically includes protocols which have only a marginal effect on the subject.

In one broad aspect, the invention discloses the use of a flowable composition comprising an organic material, capable of being cured or polymerized into a solid substance in vivo, said solid substance having an elastic modulus of at least about 100 MPa, wherein at least about 50% of said solid substance is bioresorbed or biodegraded within 10 years from the curing or polymerization, for treatment of bone, joint, and intervertebral disc defects.

In different embodiments of the invention, a variety of different materials can be used. Generally, these materials can be classified as elastomers, hydrogels, or rigid polymers. Suitable examples of elastomers include, without limitations, silicone elastomers, polyurethane elastomers, silicone-polyurethane co-polymers, polyolefin rubbers, butyl rubbers, or any combination thereof.

Suitable examples of hydrogels include, without limitations, polysaccharides, proteins, polyphosphosphazenes, poly(oxyethylene)-poly(oxypropylene) block polymers, poly(oxyethylene)-poly(oxypropylene) block polymers of ethylene diamine, poly(acrylic acids), poly(methacrylic acids), copolymers of acrylic acid and methacrylic acid, poly(vinyl acetate), sulphonated polymers, poly(N-vinyl-2-pyrrolidone), polyethylene glycol, polyethyleneoxide, poly(2-hydroxy ethyl methacrylate), copolymers of acrylates with N-vinyl pyrrolidone, N-vinyl lactams, and combinations thereof.

Suitable rigid polymers include, without limitations, polymethylmethacrylate, silicones, polyurethanes, polyvinyl alcohol, polyamides, aromatic polyamide, polyethers, polyester liquid crystal polymers, ionomers, poly(ethylene-co-methacrylic) acids, polybutylene terephtalate (PBT), polycarbonates, polyaminocarbonates, lactic acid, glycolic acid, lactide-co-glycolides, anhydrides, orthoesters, caprolactone, epoxy, and any combinations thereof.

The curing conditions may be designed so that the organic material is cured or polymerized into a porous, non-porous or essentially non-porous structure. For example, the organic materials can be cured into porous structures by co-administering these materials with porogens, such as, for example, sodium chloride, sugar, or any other components that aid in forming amorphous glasses soluble in a physiological fluid, for example, saline. A person of the ordinary skill in the art will appreciate that as the crystals of sodium chloride are dissolved in the interstitial fluid surrounding the polymerized or cured material (i.e., the solid substance), pores of the size of the crystals will be formed in that material.

The polymerization of the organic material may be initiated by the application of energy. The energy source is not important for the instant invention: it can be light energy, heat energy, radiation energy, electrical energy, mechanical energy, and any combination thereof.

Upon activation, the organic material may undergo progressive polymerization with increasing viscosity and, most likely, heat release due to exothermic reaction. In different embodiments of the invention, the peak temperature of the polymerization is not higher than 75° C., preferably not higher than 60° C., preferably not higher than 50° C. per volume of the administered composition.

In another embodiment, the flowable composition comprises at least two components. In different embodiments of the invention the components include a polymerizable or a curable material and a bioresorbable material. Examples of bioresorbable materials include, but are not limited to, oligomers, polymers, or a combination thereof of any member of the group consisting of lactic acid, glycolic acid, lactide-co-glycolides, anhydrides, orthoesters, caprolactone, and tyrosin-polycarbonate. Examples of polymerizing or crosslinking agents include, but are not limited to, monomers, oligomers, polymers, or combinations thereof of any members of the group consisting of an isocyanate-containing compound, an aldehydes-containing compound, a vinyl alcohol-containing compound, a polyol-containing compound, polyurethane, silicone, acrylic acid, cyanoacrylate, methacrylate, epoxy, and/or any combinations thereof. Thus, for example, in one embodiment the invention comprises monomers and oligomers of a bioresorbable material, such as lactic acid and/or glycolic acid and/or anhydrides thereof, and monomers and oligomers of a curable or polymerizable material such as silicone and/or polyurethane. In such embodiment, the polymerized solid substance will comprise intermixed units of lactic acid, glycolic acid, polyurethane and silicone.

A person of ordinary skill in the art will appreciate that silicone and/or polyurethane will provide the solid substance with improved mechanical properties, such as Young's modulus and ability to bear weight. On the other hand, the bioresorbable part, formed by the lactic acid and/or glycolic acid and/or anhydrides thereof will make the solid substance bioresorbable. The solid body will degrade thus reducing polyurethane and/or silicone to monomers or oligomers from polymers. The monomers and oligomers of silicone and/or polyurethane can later be excreted from the body. One exemplary embodiment of the invention is shown in FIG. 1A. In that embodiment, the bioresorbable component is a polymer of lactic acid and the polymerizable component is silicone. Upon reaction between these compounds, solid polymers will be formed. Depending on the ratios between the lactic acid and the silicone, the solid polymers will have different structures. The structural formula of one possible resulting solid body is shown when the ratio between the lactic acid polymer and the silicone compound is approximately 1:1.

The resulting solid body will be biodegraded by breaking links between the residues of lactic acid. The degradation products comprising silicone will be excreted from the patient's body.

In another embodiment, the bioresorbable compound is a monomer and/or a dimmer of glycolic acid and the curable or polymerizable compound comprises an isocyanate functional group —N═C═O. A suitable example of such isocyanate-containing compound is di-isocyanate having a general formula OCN—(CH₂)ₓ—NCO. Isocyanates are known to react with compounds having hydroxy groups to form polyurethanes which are defined by a functional group —NH(CO)O—. A person of the ordinary skill in the art will appreciate that polymer having a structural formula according to FIG. 1B will result from a reaction of a monomer and a dimer of glycolic acid with di-isocyanate. A person of the ordinary skill in the art will appreciate that components containing more than two molecules of glycolic acid can be used in this reaction. Other components containing more than two isocyanate groups may also be used for further cross-linking. Further, other compounds described above, such as, for example, polyorthoesters can be used instead of or in addition to glycolic acid.

In another embodiment of the invention, the solid substance of the invention may be prepared from the flowable composition comprising lactide with ε-caprolactone as described in details in U.S. Pat. No. 5,278,202, incorporated herein by reference in its entirety.

The components may be mixed together before (e.g., between about 2 seconds and about two minutes, preferably, between about 30 seconds and 1 minute) or during the administering of the flowable composition comprising the organic material into the desired area. It is also preferred that the curing should occur not less than 1 minute from start of mixing, more preferably not less than 3 minutes, most preferably not less than 5 minutes.

Curing by polymerization or crosslinking ensures dimensional stability of the bioresorbable polymer during resorption by creating a scaffolding network of the “linked” biopolymers. For example, bioresorbable materials can by polymerized or crosslinked by light activated free radical polymerization. In this process, a light activated free radical polymerization initiator, a non-limiting example being 2,2-dimethoxy-2-phenylacetophenone or a combination of ethyl eosin, is irradiated with long UV radiation, as disclosed in details in U.S. Pat. No. 5,879,713, incorporated herein by reference in its entirety. The initiating free radicals will add to carbon-carbon double bonds to produce carbon radicals on the bioresorbable polymers, which will further react with other carbon radicals formed on other bioresorbable polymers to form the “linked” biopolymers.

The characteristics of the flowable composition comprising the organic material and the solid structure resulting from the curing or polymerizing this organic material (such as, for example, mechanical properties, Young's modulus and/or porosity) should be selected based on the nature of the defect and the method of administration.

For example, in one broad aspect, the flowable composition comprising the organic materials of the instant invention are used for treating a patient having a fractured vertebra or a fusion stabilization of a facet joint between two vertebrae.

In this aspect of the invention, it is desirable that the solid structure resulting from the curing of the organic material should withstand significant pressure and promote ingrowth of the osseous tissue into the structure. Accordingly, it would be beneficial to select the organic material and the curing conditions in such as way as to create a rigid polymer having pores which provide a suitable environment for the growth of the new tissue inside the solid structure.

In some embodiments within this aspect of the invention, the flowable composition comprising the organic material can be delivered by an injection, such as, for example, a percutaneous injection or an intradiscal injection. Different delivery devices are suitable for this method of administration, suitable non-limiting examples being a needle or a cannula connected to a reservoir containing the composition. Suitable reservoirs include, without limitation, a syringe and a pump.

Since it is desirable to prevent additional trauma of the tissues, the delivery device should have a relatively small cross-section, such as, for example, 8 G or smaller, or more preferably 12 G or smaller. Accordingly, in this embodiment of the invention, it is beneficial if the flowable composition comprising the organic material is sufficiently fluid to be delivered by this mode of administration. In another embodiment, the composition can be delivered during a surgery to the exposed tissues. Accordingly, the composition may be sprayed, aerosol-sprayed or brushed onto the target areas (e.g., the facet joint or the site of the fracture). For this embodiment of the invention, the flowability of the composition is not crucial.

In another broad aspect of the invention, the solid structure resulting from the curing or polymerization of the organic material can be used to separate two or more tissues. For example, two tissues or organs, which are separated in a healthy patient, may become connected by a connective tissue (e.g., scar tissue) during healing after the surgery. Such connections between the tissues are generally undesirable and may cause incorrect formation of neural and vascular pathways as well as feeling of pain and discomfort when the patient moves and the organs or tissues are pulled apart. In this aspect, the composition may be applied to the tissues which need to be kept separated, by such method as, for example, spraying, misting, aerosol-spraying, brushing, or squirting those tissues with the composition of the present invention.

In another broad aspect, one of the tissues is an implant, such as, for example, a depot. Sometimes, the surgeon needs to re-position or remove the implant after a certain period. It would be understood by a person of the ordinary skill in the art that the tissues surrounding the implant may attach or grow into the implant, thus causing additional unnecessary trauma upon re-positioning and/or removal of the implant. Accordingly, one embodiment of the instant invention provides a method of preventing adhesion and ingrowth of the tissues into the implant. A person of the ordinary skill in the art will understand that the composition could be delivered to the implant, the surrounding tissues or both. In addition to the methods disclosed above, the implant may be soaked in the composition of the present invention prior to the implantation. Preferably, the implant is soaked between 2 seconds and 2 minutes prior to the implantation, more preferably, between 30 seconds and 1 minute prior to the implantation.

In another aspect, the flowable compositions comprising the organic material may be used for filling inflatable implants. Thus, in one embodiment of the instant invention, an inflatable implant, such as, for example, an intervertebral disc implant, in its deflated form may be placed into the target area, such as an intervertebral disc space, through a delivery instrument such as, for example, a hollow tube. After the correct placement of the implant is verified, the implant may be filled with the composition of the resent invention, which can be delivered via a delivery device, such as, for example, a catheter, until the implant reaches a desired volume. After filling the implant and waiting until the composition polymerizes or cures into the solid structure, the delivery device can be withdrawn. Alternatively, the opening in the implant may be fastened by a suture, a staple, blocking implant, or other sort of a fastening device.

In yet another broad aspect, the composition of the present invention may be used for tissue bulking or augmentation, such as, for example, in a combination with implant designed to fill a void in the tissue. When an implant is positioned into a tissue defect, there often is a residual void between the implant and the walls of the defect. Thus, the flowable composition may be used as a filler of that residual void.

In one aspect, the implant is non-inflatable, such as, for example, a screw. In one embodiment, the screw has a channel with openings into the possible residual void. Thus, the delivery device, such as a needle connected to the reservoir containing the composition of the instant invention, may be inserted into the channel. After the screw is placed into the defect, the practitioner may administer the composition of the instant invention, which will flow into and fill the residual void.

Other suitable non-limiting examples of the implants include cages, bladders, balloons, pouches, nucleus pulposus implants, intervertebral disc implants, corpectomy devices cervical plates, lumbar plates, anterior spinal rods, posterior spinal rods, screws, pins, intervertebral spacers, interspinous spacers, and facet implants. More specific examples of the suitable devices and method of implantation of these devices are disclosed, for example, in U.S. Patent Publications 20060186471, 20050267577, 20040133280, 20040102774, and 20040054414, all of which are incorporated herein by reference.

In one embodiment, the suitable implant comprises a biodegradable and/or semi-permeable or bioresorbable outer shell in order to allow resorption of the injected material. Further, a person of the ordinary skill in the art would appreciate that it is preferred if the outer shell of the device is flexible and can stretch without tearing when filled with the composition of the present invention.

The suitable materials for the outer shell include elastic materials, such as elastomeric materials, hydrogels, or other hydrophilic polymers, or composites thereof. Suitable elastomers include silicone, polyurethane, copolymers of silicone and polyurethane, polyolefins, such as polyisobutylene rubber and polyisoprene rubber, neoprene rubber, nitrile rubber, vulcanized rubber and combinations thereof. The vulcanized rubber described herein may be produced, for example, by a vulcanization process utilizing a copolymer produced as described, for example, in U.S. Pat. No. 5,245,098 from 1-hexene and 5-methyl-1,4-hexadiene.

Suitable hydrogels include natural hydrogels, and those formed from polyvinyl alcohol, acrylamides such as polyacrylic acid and poly (acrylonitrile-acrylic acid), polyurethanes, polyethylene glycol, poly(N-vinyl-2-pyrrolidone), acrylates such as poly(2-hydroxy ethyl methacrylate) and copolymers of acrylates with N-vinyl pyrrolidone, N-vinyl lactams, acrylamide, polyurethanes and polyacrylonitrile, or may be other similar materials that form a hydrogel. The hydrogel materials my further be cross-linked to provide further strength to the implant. Examples of polyurethanes include thermoplastic polyurethanes, aliphatic polyurethanes, segmented polyurethanes, hydrophilic polyurethanes, polyetherurethane, polycarbonate-urethane and silicone polyetherurethane.

Other suitable hydrophilic polymers include naturally occurring materials such as glucomannan gel, hyaluronic acid, polysaccharides, such as cross-linked carboxyl-containing polysaccharides, and combinations thereof.

For example, in one embodiment shown in FIG. 2, the composition of the present invention may be used to fill a bone void in a vertebra by an implantable device 10. The device 10 may be comprised of a plurality of materials as disclosed above. In one particular embodiment, the device 10 is made of a co-polymer of lactic and glycolic acids. In one embodiment, the implantable device 10 is threaded and further comprises a channel 12 along its longitudinal axis and several openings 14 which provide fluid communication between the channel 12 and the environment outside of the device 10. A person of the ordinary skill in the art will appreciate that the number of the openings 14 is not crucial for the proper work of the device 10. The number of the openings may be selected based on the mechanical properties of the material of the device 10 and the flowability of the composition 16. The composition 16 may be pre-mixed as disclosed above and the composition 16 is located within a reservoir 18, which, in the instant embodiment is a part of a delivery device (e.g., a syringe) 20. The syringe 20 further comprises a needle 22, the needle having a diameter allowing a tight fitting of the needle 22 into the channel 12. The syringe 20 may further comprise an activation area 24. As the plunger 26 of the syringe 20 pushes the composition 16, the composition 16 travels through the activation area 24 where energy is applied to the composition. As was discussed above, the source of energy is not important. In the selected embodiment, the energy is a light energy emitted from an LED.

Thus, the device 10 may be screwed into the bone void defect 30 having walls 32. It is important to note that when the device 10 is placed within the defect 30, a residual void will often be formed between the walls 32 of the bone void defect 30 and the device 10. Upon placement of the device, the needle 22 is placed into the channel 12 of the device 10, and the composition is delivered to the channel 12 by push of the plunger 26. As discussed above, the composition will be activated by the time it reaches channel 12. The composition 16 will fill the residual void due to openings 14 and high flowability of the composition 16. After the residual void is filled (which can be verified if the composition 16 comprises a radiocontrast marker), the surgeon may let the composition to cure and then the needle will be withdrawn from the channel 12.

In different embodiments of the invention, the flowable composition comprising the organic material may further comprise at least one additive. Suitable non-limiting examples of additives include, without limitation, but not limited to, analgesics, anesthetics, antibiotics, anti-inflammatories, radiocontrast media, and the like.

Growth factors can be generally suited to promote the formation of tissues, especially of the type(s) naturally occurring as components of an intervertebral disc. For example, the growth factor can promote the growth or viability of tissue or cell types occurring in the nucleus pulposus, such as nucleus pulposus cells and chondrocytes, as well as space filling cells, such as fibroblasts and connective tissue cells, such as ligament and tendon cells. Further, the growth factor can promote the growth or viability of tissue types occurring in the annulus fibrosis, as well as space filling cells, such as fibroblasts and connective tissue cells, such as ligament and tendon cells.

Exemplary growth factors include, without limitation, transforming growth factor-β (TGF-β) or a member of the TGF-β superfamily, fibroblast growth factor (FGF) or a member of the FGF family, platelet derived growth factor (PDGF) or a member of the PDGF family, a member of the hedgehog family of proteins, interleukin, insulin-like growth factor (IGF) or a member of the IGF family, colony stimulating factor (CSF) or a member of the CSF family, growth differentiation factor (GDF), cartilage derived growth factor (CDGF), cartilage derived morphogenic proteins (CDMP), bone morphogenetic protein (BMP), or any combination thereof. In particular, an exemplary growth factor includes transforming growth factor P protein, bone morphogenetic protein, fibroblast growth factor, platelet-derived growth factor, insulin-like growth factor, or any combination thereof.

In addition to growth factors suitable anti-inflammatory compounds may be added to reduce inflammation that may arise with the introduction of the composition into a vertebra fracture, a degenerating disc, or a degenerating facet joint, or as a post-surgical complication. Anti-inflammatory compounds include both steroidal and non-steroidal structures.

Suitable non-limiting examples of steroidal anti-inflammatory compounds are corticosteroids such as hydrocortisone, cortisol, hydroxyltriamcinolone, alpha-methyl dexamethasone, dexamethasone-phosphate, beclomethasone dipropionates, clobetasol valerate, desonide, desoxymethasone, desoxycorticosterone acetate, dexamethasone, dichlorisone, diflorasone diacetate, diflucortolone valerate, fluadrenolone, fluclorolone acetonide, fludrocortisone, flumethasone pivalate, fluosinolone acetonide, fluocinonide, flucortine butylesters, fluocortolone, fluprednidene (fluprednylidene) acetate, flurandrenolone, halcinonide, hydrocortisone acetate, hydrocortisone butyrate, methylprednisolone, triamcinolone acetonide, cortisone, cortodoxone, flucetonide, fludrocortisone, difluorosone diacetate, fluradrenolone, fludrocortisone, diflurosone diacetate, fluocinolone, fluradrenolone acetonide, medrysone, amcinafel, amcinafide, betamethasone and the balance of its esters, chloroprednisone, chlorprednisone acetate, clocortelone, clescinolone, dichlorisone, diflurprednate, flucloronide, flunisolide, fluoromethalone, fluperolone, fluprednisolone, hydrocortisone valerate, hydrocortisone cyclopentylpropionate, hydrocortamate, meprednisone, paramethasone, prednisolone, prednisone, beclomethasone dipropionate, triamcinolone. Mixtures of the above steroidal anti-inflammatory compounds can also be used.

Non-limiting example of non-steroidal anti-inflammatory compounds include nabumetone, celecoxib, etodolac, nimesulide, apasone, gold, oxicams, such as piroxicam, isoxicam, meloxicam, tenoxicam, sudoxicam, and CP-14,304; the salicylates, such as aspirin, disalcid, benorylate, trilisate, safapryn, solprin, diflunisal, and fendosal; the acetic acid derivatives, such as diclofenac, fenclofenac, indomethacin, sulindac, tolmetin, isoxepac, furofenac, tiopinac, zidometacin, acematacin, fentiazac, zomepirac, clindanac, oxepinac, felbinac, and ketorolac; the fenamates, such as mefenamic, meclofenamic, flufenamic, niflumic, and tolfenamic acids; the propionic acid derivatives, such as ibuprofen, naproxen, benoxaprofen, flurbiprofen, ketoprofen, fenoprofen, fenbufen, indopropfen, pirprofen, carprofen, oxaprozin, pranoprofen, miroprofen, tioxaprofen, suprofen, alminoprofen, and tiaprofenic; and the pyrazoles, such as phenylbutazone, oxyphenbutazone, feprazone, azapropazone, and trimethazone.

The variety of compounds encompassed by this group are well-known to those skilled in the art. For detailed disclosure of the chemical structure, synthesis, side effects, etc. of non-steroidal anti-inflammatory compounds, reference may be had to standard texts, including Anti-inflammatory and Anti-Rheumatic Drugs, K. D. Rainsford, Vol. I-III, CRC Press, Boca Raton, (1985), and Anti-inflammatory Agents, Chemistry and Pharmacology 1, R. A. Scherrer, et al., Academic Press, New York (1974), each incorporated herein by reference.

Mixtures of these non-steroidal anti-inflammatory compounds may also be employed, as well as the pharmacologically acceptable salts and esters of these compounds.

In addition, so-called “natural” anti-inflammatory compounds are useful in methods of the disclosed invention. Such compounds may suitably be obtained as an extract by suitable physical and/or chemical isolation from natural sources (e.g., plants, fungi, by-products of microorganisms). Suitable non-limiting examples of such compounds include candelilla wax, alpha bisabolol, aloe vera, Manjistha (extracted from plants in the genus Rubia^{, particularly} Rubia Cordifolia), and Guggal (extracted from plants in the genus Commiphora, particularly Commiphora Mukul), kola extract, chamomile, sea whip extract, compounds of the Licorice (the plant genus/species Glycyrrhiza glabra) family, including glycyrrhetic acid, glycyrrhizic acid, and derivatives thereof (e.g., salts and esters). Suitable salts of the foregoing compounds include metal and ammonium salts. Suitable esters include C₂-C₂₄ saturated or unsaturated esters of the acids, preferably C₁₀-C₂₄, more preferably C₁₆-C₂₄. Specific examples of the foregoing include oil soluble licorice extract, the glycyrrhizic and glycyrrhetic acids themselves, monoammonium glycyrrhizinate, monopotassium glycyrrhizinate, dipotassium glycyrrhizinate, 1-beta-glycyrrhetic acid, stearyl glycyrrhetinate, and 3-stearyloxy-glycyrrhetinic acid, and disodium 3-succinyloxy-beta-glycyrrhetinate.

Suitable antibiotics include, without limitation nitroimidazole antibiotics, tetracyclines, penicillins, cephalosporins, carbopenems, aminoglycosides, macrolide antibiotics, lincosamide antibiotics, 4-quinolones, rifamycins and nitrofurantoin. Suitable specific compounds include, without limitation, ampicillin, amoxicillin, benzylpenicillin, phenoxymethylpenicillin, bacampicillin, pivampicillin, carbenicillin, cloxacillin, cyclacillin, dicloxacillin, methicillin, oxacillin, piperacillin, ticarcillin, flucloxacillin, cefuroxime, cefetamet, cefetrame, cefixine, cefoxitin, ceftazidime, ceftizoxime, latamoxef, cefoperazone, ceftriaxone, cefsulodin, cefotaxime, cephalexin, cefaclor, cefadroxil, cefalothin, cefazolin, cefpodoxime, ceftibuten, aztreonam, tigemonam, erythromycin, dirithromycin, roxithromycin, azithromycin, clarithromycin, clindamycin, paldimycin, lincomycirl, vancomycin, spectinomycin, tobramycin, paromomycin, metronidazole, tinidazole, ornidazole, amifloxacin, cinoxacin, ciprofloxacin, difloxacin, enoxacin, fleroxacin, norfloxacin, ofloxacin, temafloxacin, doxycycline, minocycline, tetracycline, chlortetracycline, oxytetracycline, methacycline, rolitetracyclin, nitrofurantoin, nalidixic acid, gentamicin, rifampicin, amikacin, netilmicin, imipenem, cilastatin, chloramphenicol, furazolidone, nifuroxazide, sulfadiazin, sulfametoxazol, bismuth subsalicylate, colloidal bismuth subcitrate, gramicidin, mecillinam, cloxiquine, chlorhexidine, dichlorobenzylalcohol, methyl-2-pentylphenol or any combination thereof.

Suitable analgesics include, without limitation, non-steroid anti-inflammatory drugs, non-limiting examples of which have been recited above. Further, analgesics also include other types of compounds, such as, for example, opioids (such as, for example, morphine and naloxone), local anaesthetics (such as, for example, lidocaine), glutamate receptor antagonists, α-adrenoreceptor agonists, adenosine, canabinoids, cholinergic and GABA receptors agonists, and different neuropeptides. A detailed discussion of different analgesics is provided in Sawynok et al., (2003) Pharmacological Reviews, 55:1-20, the content of which is incorporated herein by reference.

In other embodiments, the at least one additive may include compounds suppressing cell motility and cell attachment, such as, for example, amphiphysin or endostatin. Otsuka et al., Biochem Biophys Res Commun., 301(3):769-75 (2003); Furumatsu et al., J Biochem (Tokyo), 131(4):619-26 (2002).

The at least one additive may also include a radiocontrast agent to verify the placement and/or the distribution of the composition in the target area. Suitable radiocontrast agents include barium and iodine compounds, metal ions, nitroxides, and gadolinium complexes, such as gadodiamine.

Further, the at least one additive may comprise cells, such as, for example, stem cells, especially autologous stem cells, including, without limitation, bone marrow stem cells and mesenchymal stem cells.

A person of the ordinary skill in the art will appreciate that the additives may be formulated into sustained-release formulations, such as, for example, microspheres comprising a biodegradable polymer. Suitable examples of the biodegradable polymers suitable for the present invention include but are not limited to poly(alpha-hydroxy acids), poly(lactide-co-glycolide) (PLGA), polylactide (PLA), polyglycolide (PG), polyethylene glycol (PEG) conjugates of poly(alpha-hydroxy acids), polyorthoesters, polyaspirins, polyphosphagenes, collagen, starch, chitosans, gelatin, alginates, dextrans, vinylpyrrolidone, polyvinyl alcohol (PVA), PVA-g-PLGA, PEGT-PBT copolymer (polyactive), methacrylates, poly(N-isopropylacrylamide), PEO-PPO-PEO (pluronics), PEO-PPO-PAA copolymers, PLGA-PEO-PLGA, or combinations thereof.

In yet another embodiment, the at least one additive may further comprise a biomaterial. Suitable biomaterials include, without limitation, different polymers, metals or ceramics. Examples of ceramics include calcium phosphate, hydroxyapatite, calcium sulfate, bioactive glass or a combination thereof.

The choice and the formulation of the additives which can be included into the composition of the present invention depend on the application of the composition of the instant invention. For example, if the composition is used to prevent tissue adhesion or ingrowth, compounds which inhibit cell motility and cell attachment can be used. On the other hand, if the composition is used for the application where tissue ingrowth is desired (such as, for example, vertebral fracture of fusion stabilization of facet joints), compounds which inhibit cell motility and cell attachment are not desired. Instead, the use of growth factors, such as, for example BMPs, and more specifically, BMP-2 or BMP-7 is more advantageous. Further, cells, such as, for example, stem cells, including without limitation, autologous bone marrow cells, can also be used.

The patent and scientific literature referred to herein establishes the knowledge that is available to those with skill in the art. All U.S. patents and published or unpublished U.S. patent applications cited herein are incorporated by reference. All published foreign patents and patent applications cited herein are hereby incorporated by reference. All other published references, documents, manuscripts and scientific literature cited herein are hereby incorporated by reference.

While this invention has been particularly shown and described with references to preferred embodiments thereof, it will be understood by those skilled in the art that various changes in form and details may be made therein without departing from the scope of the invention encompassed by the claims.

1. A method of treating a patient having a fractured vertebra comprising administering to said fractured vertebra a flowable composition comprising an organic material, capable of being cured or polymerized into a solid substance in vivo, said solid substance having an elastic modulus of at least about 100 MPa, wherein at least about 50% of said solid substance is bioresorbed or biodegraded within 10 years from the curing or polymerization.

2. The method of claim 1, wherein said flowable composition is capable of being administered through a delivery device comprising a channel having a cross-section not larger than about 8 G.

3. The method of claim 2, wherein the delivery device is a needle or a cannula.

4. The method of claim 1, wherein a fracture of the fractured vertebra is a compression fracture.

5. The method of claim 1, wherein the flowable composition comprises a monomer, an oligomer, a polymer, or a combination thereof of any member of the group consisting of lactic acid, glycolic acid, lactide-co-glycolides, anhydrides, orthoesters, caprolactone, and tyrosin-polycarbonate.

6. The method of claim 1, wherein the flowable composition comprises a monomer, an oligomer, a polymer, or a combination thereof of any member of the group consisting of an isocyanate-containing compound, an aldehydes-containing compound, a vinyl alcohol-containing compound, a polyol-containing compound, polyurethane, silicone, acrylic acid, cyanoacrylate, methacrylate, epoxy, and any combination thereof.

7. The method of claim 1, wherein the flowable composition comprises at least two components.

8. The method of claim 7, wherein:
at least one of the at least two components comprises a monomer, an oligomer, a polymer, or a combination thereof of any member of the group consisting of lactic acid, glycolic acid, lactide-co-glycolides, anhydrides, orthoesters, caprolactone, tyrosin-polycarbonate, and any combination thereof; and
at least a second of the at least two components comprises a monomer, an oligomer, a polymer, or a combination thereof of any member of the group consisting of an isocyanate-containing compound, an aldehydes-containing compound, a vinyl alcohol-containing compound, a polyol-containing compound, polyurethane, silicone, acrylic acid, cyanoacrylate, methacrylate, epoxy, and any combination thereof.

9. The method of claim 7, wherein said organic material comprises:
a monomer or an oligomer of lactic acid; and
a monomer or an oligomer of silicone or polyurethane.

10. The method of claim 7, wherein a mixing of the at least two components initiates curing.

11. The method of claim 7, wherein the at least two components of the flowable composition are mixed no more than about 2 minutes prior to administering the flowable composition.

12. The method of claim 1, wherein the curing or polymerizing of the organic material is activated by an application of energy.

13. The method of claim 12, wherein the energy is selected from the group consisting of light energy, heat energy, radiation energy, electrical energy, mechanical energy and any combination thereof.

14. The method of claim 13, wherein the energy is applied before, during, or after administering the flowable composition.

15. The method of claim 1, wherein the polymerization of the organic material has a peak temperature not higher than approximately 75° C. per volume of the administered flowable composition.

16. The method of claim 1, wherein the flowable composition further comprises at least one additive.

17. The method of claim 16, wherein the at least one additive is selected from the group consisting of growth factors, analgetics, anesthetics, antibiotics, anti-inflammatory agents, radiocontrast agents, a biomaterial, and any combinations thereof.

18. The method of claim 17, wherein the at least one additive is selected from the group consisting of BMP-2 and LMP-1.

19. The method of claim 17, wherein the radiocontrast agent is a gadodiamine based radiocontrast agent.

20. The method of claim 16, wherein the at least one additive is in a sustained-release formulation.

21. The method of claim 1, wherein the solid substance is porous.

22. A method of treating a patient having a back pain caused by a degenerative facet joint or a degenerative disc disease comprising administering into the degenerative facet joint or the degenerating disc a flowable composition comprising an organic material, capable of being cured or polymerized into a solid substance in vivo, said solid substance having an elastic modulus of at least about 1 MPa, wherein at least about 50% of said solid substance is bioresorbed or biodegraded within 10 years from the curing or polymerization.

23. The method of claim 22, wherein said flowable composition is administered by an injection.

24. The method of claim 23, wherein the injection is a percutaneous injection.

25. The method of claim 22, wherein said flowable composition is capable of being administered through a delivery device comprising a channel having a cross-section not larger than about 12 G.

26. The method of claim 25, wherein the delivery device is a needle or a cannula.

27. The method of claim 22, wherein the flowable composition comprises a monomer, an oligomer, a polymer, or a combination thereof of any member of the group consisting of lactic acid, glycolic acid, lactide-co-glycolides, anhydrides, orthoesters, caprolactone, and tyrosin-polycarbonate.

28. The method of claim 22, wherein the flowable composition comprises a monomer, an oligomer, a polymer, or a combination thereof of any member of the group consisting of an isocyanate-containing compound, an aldehydes-containing compound, a vinyl alcohol-containing compound, a polyol-containing compound, polyurethane, silicone, acrylic acid, cyanoacrylate, methacrylate, epoxy, and any combination thereof.

29. The method of claim 22, wherein the flowable composition comprises at least two components.

30. The method of claim 29, wherein:
at least one of the at least two components comprises a monomer, an oligomer, a polymer, or a combination thereof of any member of the group consisting of lactic acid, glycolic acid, lactide-co-glycolides, anhydrides, orthoesters, caprolactone, tyrosin-polycarbonate, and any combination thereof; and
at least a second of the at least two components comprises a monomer, an oligomer, a polymer, or a combination thereof of any member of the group consisting of an isocyanate-containing compound, an aldehydes-containing compound, a vinyl alcohol-containing compound, a polyol-containing compound, polyurethane, silicone, acrylic acid, cyanoacrylate, methacrylate, epoxy, and any combination thereof.

31. The method of claim 29, wherein said organic material comprises:
a monomer or an oligomer of lactic acid; and
a monomer or an oligomer of silicone or polyurethane.

32. The method of claim 29, wherein a mixing of the at least two components initiates curing.

33. The method of claim 29, wherein the at least two components of the flowable composition are mixed no more than about 2 minutes prior to administering the flowable composition.

34. The method of claim 22, wherein the curing or polymerizing of the organic material is activated by an application of energy.

35. The method of claim 34, wherein the energy is selected from the group consisting of light energy, heat energy, radiation energy, electrical energy, mechanical energy and any combination thereof.

36. The method of claim 34, wherein the energy is applied before, during, or after administering the flowable composition.

37. The method of claim 22, wherein the polymerization of the organic material has a peak temperature not higher than approximately 75° C. per volume of the administered flowable composition.

38. The method of claim 22, wherein said flowable composition is administered for a non-fusion stabilization of the degenerative facet joint or the degenerating disc.

39. The method of claim 38, wherein the flowable composition further comprises at least one additive.

40. The method of claim 39, wherein the at least one additive is selected from the group consisting of analgetics, anesthetics, antibiotics, anti-inflammatory agents, radiocontrast agents, agents preventing cell motility, a biomaterial, and any combinations thereof.

41. The method of claim 40, wherein the at least one additive is selected from the group consisting of BMP-2 and LMP-1.

42. The method of claim 40, wherein the radiocontrast agent is a gadodiamine based radiocontrast agent.

43. The method of claim 39, wherein the at least one additive is in a sustained-release formulation.

44. The method of claim 38, wherein the solid substance is essentially non-porous.

45. The method of claim 22, wherein said flowable composition is administered for a fusion stabilization of the degenerative facet joint or the vertebrae separated by the degenerating disc.

46. The method of claim 45, wherein the flowable composition further comprises at least one additive.

47. The method of claim 46, wherein the at least one additive is selected from the group consisting of growth factors, analgetics, anesthetics, antibiotics, anti-inflammatory agents, radiocontrast agents, a biomaterial, and any combinations thereof.

48. The method of claim 47, wherein the at least one additive is selected from the group consisting of BMP-2 and LMP-1.

49. The method of claim 47, wherein the radiocontrast agent is a gadodiamine based radiocontrast agent.

50. The method of claim 45, wherein the solid substance is porous.

51. The method of claim 45, wherein the solid substance has a Young's modulus of at least about 50 MPa.

52. The method of claim 22, wherein no tissue is removed prior to the administering of the composition.

53. A method of preventing an adhesion of a first tissue to at least a second tissue after a surgery comprising administering to the first tissue a flowable composition comprising an organic material, capable of being cured or polymerized into a film in vivo, said film having an elastic modulus of at least about 50 MPa, wherein at least about 50% of said film is bioresorbed or biodegraded within 10 years from the curing or polymerization.

54. The method of claim 53, wherein the first tissue is an implant.

55. The method of claim 53, wherein the flowable composition is administered by spraying, misting, aerosol-spraying, brushing, squirting, soaking or a combination thereof.

56. The method of claim 53, further comprising administering the flowable composition to the at least the second tissue.

57. The method of claim 53, wherein the film is non-porous or essentially non-porous.

58. The method of claim 53, wherein the flowable composition comprises a monomer, an oligomer, a polymer, or a combination thereof of any member of the group consisting of lactic acid, glycolic acid, lactide-co-glycolides, anhydrides, orthoesters, caprolactone, and tyrosin-polycarbonate.

59. The method of claim 53, wherein the flowable composition comprises a monomer, an oligomer, a polymer, or a combination thereof of any member of the group consisting of an isocyanate-containing compound, an aldehydes-containing compound, a vinyl alcohol-containing compound, a polyol-containing compound, polyurethane, silicone, acrylic acid, cyanoacrylate, methacrylate, epoxy, and any combination thereof.

60. The method of claim 53, wherein the flowable composition comprises at least two components.

61. The method of claim 60, wherein:
at least one of the at least two components comprises a monomer, an oligomer, a polymer, or a combination thereof of any member of the group consisting of lactic acid, glycolic acid, lactide-co-glycolides, anhydrides, orthoesters, caprolactone, tyrosin-polycarbonate, and any combination thereof; and
at least a second of the at least two components comprises a monomer, an oligomer, a polymer, or a combination thereof of any member of the group consisting of an isocyanate-containing compound, an aldehydes-containing compound, a vinyl alcohol-containing compound, a polyol-containing compound, polyurethane, silicone, acrylic acid, cyanoacrylate, methacrylate, epoxy, and any combination thereof.

62. The method of claim 60, wherein said organic material is wherein said organic material comprises:
a monomer or an oligomer of lactic acid; and
a monomer or an oligomer of silicone or polyurethane.

63. The method of claim 60, wherein a mixing of the at least two components initiates curing.

64. The method of claim 60, wherein the at least two components of the flowable composition are mixed no more than about 2 minutes prior to administering the flowable composition.

65. The method of claim 53, wherein the curing or polymerizing of the organic material is activated by an application of energy.

66. The method of claim 65, wherein the energy is selected from the group consisting of light energy, heat energy, radiation energy, electrical energy, mechanical energy and any combination thereof.

67. The method of claim 65, wherein the energy is applied before, during, or after administering the flowable composition.

68. The method of claim 53, wherein the polymerization of the organic material has a peak temperature not higher than approximately 75° C. per volume of the administered flowable composition.

69. The method of claim 53, wherein the flowable composition further comprises at least one additive.

70. The method of claim 69, wherein the at least one additive is selected from the group consisting of growth factors, analgetics, anesthetics, antibiotics, anti-inflammatory agents, radiocontrast agents, a biomaterial, and any combinations thereof.

71. The method of claim 70, wherein the at least one additive is an anti-inflammatory agent.

72. The method of claim 70, wherein the radiocontrast agent is a gadodiamine based radiocontrast agent.

73. The method of claim 69, wherein the at least one additive is in a sustained-release formulation.

74. A method of tissue augmentation comprising: administering to a tissue in need thereof a flowable composition comprising an organic material, capable of being cured or polymerized into a solid substance in vivo, said solid substance having an elastic modulus of at least about 1 MPa, wherein at least about 50% of said solid substance is bioresorbed or biodegraded within 10 years from the curing or polymerization.

75. The method of claim 74, wherein the tissue in need of augmentation comprises a tissue void formed between an implant and walls of a surrounding tissue.

76. The method of claim 74, wherein the solid substance is porous.

77. The method of claim 74, wherein said flowable composition is capable of being administered through a delivery device comprising a channel having a cross-section not larger than about 12 G.

78. The method of claim 74, wherein the delivery device is a needle or a cannula.

79. The method of claim 74, wherein the flowable composition comprises a monomer, an oligomer, a polymer, or a combination thereof of any member of the group consisting of lactic acid, glycolic acid, lactide-co-glycolides, anhydrides, orthoesters, caprolactone, and tyrosin-polycarbonate.

80. The method of claim 74, wherein the flowable composition comprises a monomer, an oligomer, a polymer, or a combination thereof of any member of the group consisting of an isocyanate-containing compound, an aldehydes-containing compound, a vinyl alcohol-containing compound, a polyol-containing compound, polyurethane, silicone, acrylic acid, cyanoacrylate, methacrylate, epoxy, and any combination thereof.

81. The method of claim 74, wherein the flowable composition comprises at least two components.

82. The method of claim 81, wherein:
at least one of the at least two components comprises a monomer, an oligomer, a polymer, or a combination thereof of any member of the group consisting of lactic acid, glycolic acid, lactide-co-glycolides, anhydrides, orthoesters, caprolactone, tyrosin-polycarbonate, and any combination thereof; and
at least a second of the at least two components comprises a monomer, an oligomer, a polymer, or a combination thereof of any member of the group consisting of an isocyanate-containing compound, an aldehydes-containing compound, a vinyl alcohol-containing compound, a polyol-containing compound, polyurethane, silicone, acrylic acid, cyanoacrylate, methacrylate, epoxy, and any combination thereof.

83. The method of claim 81, wherein said organic material comprises:
a monomer or an oligomer of lactic acid; and
a monomer or an oligomer of silicone or polyurethane.

84. The method of claim 81, wherein a mixing of the at least two components initiates curing.

85. The method of claim 81, wherein the at least two components of the flowable composition are mixed no more than about 2 minutes prior to administering the flowable composition.

86. The method of claim 74, wherein the curing or polymerizing of the organic material is activated by an application of energy.

87. The method of claim 86, wherein the energy is selected from the group consisting of light energy, heat energy, radiation energy, electrical energy, mechanical energy and any combination thereof.

88. The method of claim 86, wherein the energy is applied before, during, or after administering the flowable composition.

89. The method of claim 74, wherein the polymerization of the organic material has a peak temperature not higher than approximately 75° C. per volume of the administered flowable composition.

90. The method of claim 74, wherein the flowable composition further comprises at least one additive.

91. The method of claim 90, wherein the at least one additive is selected from the group consisting of growth factors, analgetics, anesthetics, antibiotics, anti-inflammatory agents, radiocontrast agents, a biomaterial, and any combinations thereof.

92. The method of claim 91, wherein the at least one additive is selected from the group consisting of BMP-2 and LMP-1.

93. The method of claim 91, wherein the radiocontrast agent is a gadodiamine based radiocontrast agent.

94. The method of claim 90, wherein the at least one additive is in a sustained-release formulation.

95. The method of claim 75, wherein the implant comprises a device selected from the group consisting of cages, bladders, balloons, pouches, nucleus pulposus implants, intervertebral disc implants, corpectomy devices, cervical plates, lumbar plates, anterior spinal rods, posterior spinal rods, screws, pins, intervertebral spacers, interspinous spacers, and facet implants.

96. The method of claim 74, wherein the tissue in need of augmentation is a soft tissue.

97. The method of claim 74, wherein the tissue in need of augmentation is a hard tissue.

98. The method of claim 97, wherein the Young's modulus of the solid structure is at least about 50 MPa.

99. A method of treating a tissue defect comprising:
inserting an inflatable device into the tissue defect;
inflating the inflatable device with a flowable composition comprising an organic material, capable of being cured or polymerized into a solid substance in vivo, said solid substance having an elastic modulus of at least about 5 MPa, wherein at least about 50% of said solid substance is bioresorbed or biodegraded within 10 years from the curing or polymerization.

100. The method of claim 99, wherein the inflating of the inflatable device is performed after inserting the inflatable device into the tissue defect.

101. The method of claim 99, wherein the inflatable device is selected from the group consisting of cages, bladders, balloons, pouches, nucleus pulposus implants, intervertebral disc implants, corpectomy devices, cervical plates, lumbar plates, anterior spinal rods, posterior spinal rods, screws, pins, intervertebral spacers, interspinous spacers, and facet implants.

102. The method of claim 99, wherein the inflatable device comprises a semi-permeable outer shell.

103. The method of claim 99, wherein the outer shell of the inflatable device is bioresorbable.

104. The method of claim 99, wherein said flowable composition is capable of being administered through a delivery device comprising a channel having a cross-section not larger than about 12 G.

105. The method of claim 104, wherein the delivery device is a needle or a cannula.

106. The method of claim 99, wherein the flowable composition comprises a monomer, an oligomer, a polymer, or a combination thereof of any member of the group consisting of lactic acid, glycolic acid, lactide-co-glycolides, anhydrides, orthoesters, caprolactone, and tyrosin-polycarbonate.

107. The method of claim 99, wherein the flowable composition comprises a monomer, an oligomer, a polymer, or a combination thereof of any member of the group consisting of an isocyanate-containing compound, an aldehydes-containing compound, a vinyl alcohol-containing compound, a polyol-containing compound, polyurethane, silicone, acrylic acid, cyanoacrylate, methacrylate, epoxy, and any combination thereof.

108. The method of claim 99, wherein the flowable composition comprises at least two components.

109. The method of claim 108, wherein:
at least one of the at least two components comprises a monomer, an oligomer, a polymer, or a combination thereof of any member of the group consisting of lactic acid, glycolic acid, lactide-co-glycolides, anhydrides, orthoesters, caprolactone, tyrosin-polycarbonate, and any combination thereof; and
at least a second of the at least two components comprises a monomer, an oligomer, a polymer, or a combination thereof of any member of the group consisting of an isocyanate-containing compound, an aldehydes-containing compound, a vinyl alcohol-containing compound, a polyol-containing compound, polyurethane, silicone, acrylic acid, cyanoacrylate, methacrylate, epoxy, and any combination thereof.

110. The method of claim 108, wherein said organic material comprises:
a monomer or an oligomer of lactic acid; and
a monomer or an oligomer of silicone or polyurethane.

111. The method of claim 108, wherein a mixing of the at least two components initiates curing.

112. The method of claim 108, wherein the at least two components of the flowable composition are mixed no more than about 2 minutes prior to administering the flowable composition.

113. The method of claim 99, wherein the curing or polymerizing of the organic material is activated by an application of energy.

114. The method of claim 113, wherein the energy is selected from the group consisting of light energy, heat energy, radiation energy, electrical energy, mechanical energy and any combination thereof.

115. The method of claim 113, wherein the energy is applied before, during, or after administering the flowable composition.

116. The method of claim 99, wherein the polymerization of the organic material has a peak temperature not higher than approximately 75° C. per volume of the administered flowable composition.

117. The method of claim 99, wherein the flowable composition further comprises at least one additive.

118. The method of claim 117, wherein the at least one additive is selected from the group consisting of growth factors, analgetics, anesthetics, antibiotics, anti-inflammatory agents, radiocontrast agents, a biomaterial, and any combinations thereof.

119. The method of claim 118, wherein the at least one additive is selected from the group consisting of BMP-2 and LMP-1.

120. The method of claim 118, wherein the radiocontrast agent is a gadodiamine based radiocontrast agent.

121. The method of claim 117, wherein the at least one additive is in a sustained-release formulation.
